(19) United States
(12) Patent Application Publication
(10) Pub. No.: US 2021/0236523 A1
(43) Pub. Date: Aug. 5, 2021

(54) PSYCHEDELIC TREATMENT FOR HEADACHE DISORDERS

(71) Applicants: YALE UNIVERSITY, New Haven, CT (US); The United States of America as represented by the Department of Veterans Affairs, Washington, DC (US)

(72) Inventors: Emmanuelle SCHINDLER, New Haven, CT (US); Deepak D'SOUZA, Guilford, CT (US)

(21) Appl. No.: 17/168,638

(22) Filed: Feb. 5, 2021

Related U.S. Application Data
(60) Provisional application No. 62/970,476, filed on Feb. 5, 2020.
(60) Provisional application No. 63/013,252, filed on Apr. 21, 2020.
(60) Provisional application No. 63/020,485, filed on May 5, 2020.

(51) Int. Cl. A61K 31/675; A61K 31/48; A61K 31/137; A61K 31/4045; A61K 9/00; A61P 25/06

(52) U.S. Cl. CPC A61K 31/675; A61K 31/48; A61P 25/06; A61K 31/4045; A61K 9/0053; A61K 31/137

(57) ABSTRACT

A method of treating headache disorders, by administering an effective amount of a composition of a psychedelic to the individual and treating the headache disorder. A method of treating migraine headache in an individual, by administering an effective amount of a psychedelic to the individual and reducing migraine headache burden. A method of treating cluster headache in an individual, by administering an effective amount of a psychedelic to the individual and reducing cluster headache burden. A method of treating headache disorders, by administering a single treatment of a psychedelic to an individual and providing a long term effect in preventing headaches.

### CROSS-REFERENCE TO RELATED APPLICATION

This application claims the benefit of priority to U.S. Provisional Patent Application Ser. No. 62/970,476 entitled “PSILOCYBIN TREATMENT FOR PAIN AND HEADACHE,” filed Feb. 5, 2020, the disclosure of which is incorporated herein by reference in its entirety.

### BACKGROUND OF THE INVENTION

The present invention relates to methods of treating headache disorders such as migraine and cluster headache. More specifically, the present invention relates to methods of using psychedelics in acutely treating as well as preventing headache attacks.

Headaches are paroxysmal episodes, or attacks, of head pain, which often include face and neck pain, and may be associated with a variety of neurological symptoms. Headache disorders can be classified into primary headache disorders, secondary headache disorders, cranial neuralgias, facial pain, and other headache types. Primary headache disorders include tension-type headache, migraine, and cluster headache. Secondary headache disorders are caused by an underlying pathology or injury. Some headaches can be caused by exogenous factors, such as medication overuse.

Migraine headache is a common neurological condition with top worldwide disability rating. Up to one in ten individuals who suffer from migraines does not respond well to medical treatment and lifestyle modification and suffers poor functional ability and quality of life and increased healthcare utilization. Migraine attacks recur with moderate to severe pain, and include nausea, and sensitivities to light and sound. Attacks can also include weakness, paresthesias, confusion, and vertigo. Migraine occurs in about 12 percent of individuals in the United States. They are thought to be genetic. Many different factors can trigger migraine attacks, such as stress, anxiety, hormonal changes, bright or flashing lights, loud noises, strong smells, medicines, sleep patterns, sudden weather changes, overexertion, tobacco use, caffeine or lack of caffeine, missed meals, and foods and additives.

Migraine treatments focus on aborting attacks and preventing further attacks and include medicines such as analgesics, triptans, antiepileptics, BOTOX® injections, stress management, rest, and hormone therapy. Some novel targets for migraine therapy have emerged in recent years, including the calcitonin gene-related peptide, the vagus nerve, and the 5-hydroxytryptamine (5-HT)1F receptor. While these new approaches offer promise, no single therapy is effective in all patients and a combination of treatments is often necessary to manage the disease, necessitating the continued search for new therapeutic avenues in migraine headache.

Cluster headaches are short painful headaches that occur every day for weeks or months and can occur seasonally. Their cause is unknown but involves the trigeminal nerve in the face and pain is felt behind one eye and on one side of the face. Treatment usually involves prescribing triptans (sumatriptan, zolmitriptan), dihydroergotamine, lidocaine, or oxygen. The severity of pain in cluster headache has earned the disorder the pseudonym suicide headache. This, in addition to the many limits of conventional therapy, which include poor efficacy, intolerable side effects, lack of insurance coverage, and deficiencies in clinician knowledge, have driven patients to unconventional agents and practices. Since the early 1990s, reports across social media, as well as peer-reviewed materials, suggest that psilocybin, lysergic acid diethylamide (LSD), and other indoleamine 5-hydroxytryptamine (5-HT)2A receptor agonist compounds confer sustained therapeutic benefit in cluster headache. Importantly, in contrast to conventional therapies, psilocybin and related compounds are reported to produce lasting reductions in headache burden after a single or few doses and in some cases may even induce remission. These extraordinary effects suggest a yet unknown mechanism of action that has tremendous potential value in the understanding and management of cluster headache.

Psychedelic drugs, such as psilocybin and LSD, are reported to stop acute cluster attacks, terminate cluster cycles, and induce and prolong remission from cluster headache. (Sewell, et al., 2006; Schindler et al., 2015). The nonhallucinogenic LSD derivative BOL-148 was also able to suppress attacks in cluster headache (Karst, et al., 2010). Three single oral doses of 30 μg/kg BOL-148 was found to either break a cluster headache cycle or considerably improve the frequency and intensity of attacks.

Andersson, et al. (Harm Reduct J. 2017; 14:60) reports that many migraine and cluster headache sufferers are desperate for an effective treatment and this causes them depression, anxiety, and stress disorders. In online forums many individuals report that psilocybin was effective in treating migraine and cluster headache. Self-treatment is used with so-called busting which involves a few low-moderate doses taken over a period of 1-2 weeks (between 1-2 grams dried P. cubensis taken three times, approximately 5 days apart each), frequent microdosing (taking small doses devoid of psychedelic effects repeatedly), or single large doses.

Psilocybin is metabolized to psilocin, which is an agonist for serotonin receptors, and binds to 5-HT2A with high affinity and to 5-HT1 with low affinity. Psilocin can indirectly increase concentrations of dopamine and serotonin in the brain, though it has no effect itself on the dopamine receptor. The mechanism by which psilocybin treats headache is currently unknown. While LSD and psilocybin have been used by individuals, there has been no guarantee that the dosing or even content of the substance that they were using was accurate, and there was no proper scientific observation or data gathering.

There remains a need for treating headache disorders especially for migraine and cluster headache.

### SUMMARY OF THE INVENTION

The present invention provides for a method of treating headache disorders, by administering an effective amount of a composition of a psychedelic to the individual and treating the headache disorder in the individual.

The present invention provides fora method of treating migraine headache in an individual, by administering an effective amount of a psychedelic to the individual and reducing migraine headache burden.

The present invention provides for a method of treating cluster headache in an individual, by administering an effective amount of a psychedelic to the individual and reducing cluster headache burden.

The present invention also provides for a method of treating headache disorders, by administering a single treatment of a psychedelic to an individual and providing a long term effect in preventing headaches.

### DESCRIPTION OF THE DRAWINGS

Other advantages of the present invention are readily appreciated as the same becomes better understood by reference to the following detailed description when considered in connection with the accompanying drawings wherein:

FIG. 1 shows statistics for data gathered from a survey about cluster headache;

FIG. 2 is a graph of abortive medications with number and percentage of responders who tried versus their reported efficacy;

FIG. 3 is a graph of preventive medications with number and percentage of responders who tried versus their reported efficacy;

FIG. 4 is a graph comparing medications that effectively affected the cluster period or remission period;

FIG. 5 is a graph comparing medications being taken when transforming from episodic to cluster headache;

FIG. 6 is a table of medications used and dose ranges;

FIGS. 7A-7I are graphs showing change in migraine burden in the two weeks after the administration of a single oral dose of 0.143 mg/kg psilocybin compared to placebo, FIG. 7A shows migraine headache days per week, FIG. 7B shows migraine attacks per week, FIG. 7C shows duration, FIG. 7D shows pain, FIG. 7E shows light sensitivity, FIG. 7F shows sound sensitivity, FIG. 7G shows nausea/vomiting, FIG. 7H shows functional impairment, and FIG. 7I shows days/week using a migraine abortive;

FIG. 8 is a graph showing time to first and second migraine attack after the single oral administration of 0.143 mg/kg psilocybin compared to placebo.

FIG. 9 is a graph of change in weekly cluster attacks over three weeks with a pulse regimen (3 doses about 5 days apart each) of placebo or psilocybin (0.143 mg/kg); and

FIG. 10 is a graph of change in weekly attacks over eight weeks with a pulse regimen (3 doses about 5 days apart each) of placebo or psilocybin (0.143 mg/kg).

### DETAILED DESCRIPTION OF THE INVENTION

The present invention provides generally for methods of treating headache disorders, by administering a composition of a psychedelic to the individual. The method is especially useful in treating migraine and cluster headache.

“Headache disorder” as used herein refers to disorders of the nervous system characterized by recurring headache attacks, and can include migraine, tension-type headache, cluster headache and other trigeminal autonomic cephalalgias, and secondary headache disorders including medication overuse headache, and other headache disorders listed in the ICHD 3^{rd} Edition. Headache attacks differ among headache disorders in the location, pain quality, associated symptoms, duration, and recurrence pattern. For example, migraine attacks involve pulsing or throbbing unilateral or bilateral pain, nausea, vomiting, sensitivity to light and sound, lasting hours to days, separated by days to months or years. The compositions of the present invention can be used to treat any headache disorder, either in an abortive or preventive capacity.

The psychedelics used in the methods of the present invention can be, but are not limited to, psilocybin, lysergic acid diethylamide (LSD), mescaline, dimethyltryptamine (DMT), 2,5-dimethoxy-4-iodoamphetamine (DOI), 2,5-dimethoxy-4-bromoamphetamie (DOB), salts thereof, tartrates thereof, analogs thereof, or homologues thereof. The term “psychedelic” as used herein refers to classic serotinergic psychedelics in reference to compounds that have activity at the serotonin 2A receptor.

Psilocybin (3-(2-dimethylaminoethyl)-1H-indol-4-yl] dihydrogen phosphate) is a psychedelic drug that is produced by psilocybin mushrooms, such as, but not limited to, P. azurescens, P. semilanceata, and P. cyanescens. It is converted in the body to psilocin (the active molecule) and acts as a partial agonist for serotonin 5-hydroxytryptamine (5-HT) receptors (with high affinity for 5-HT_{2B} and 5-HT_{2C}, and modest affinity to 5-HT_{2A}). Psilocybin can also indirectly increase the concentration of dopamine and serotonin in the brain. The psilocybin used in the present invention can be naturally derived or synthetic. Homologs thereof and analogs thereof can also be used. Any functional equivalents can also be used, i.e. any compound that provides the same function as psilocybin. One example of an analog is psilacetin (O-Acetylpsilocin), which is also converted to psilocin in the body. Baeocystin is another analog ([3-(2-methylaminoethyl)-1H-indol-4-yl] dihydrogen phosphate). Any of the psychedelics can work by augmenting excitatory or inhibitory neuronal activity. For example, psilocybin can diminish activity in brain regions such as the ventral medial prefrontal cortex, and decreased blood flow in the thalamus and anterior cingulate cortex.

A dose of 1 to 50 mg psilocybin can be administered orally or by any suitable method (such as intravenous) with appropriate dose conversion (for example, an IV dose will be lower than an oral dose), over any suitable time period, such as hours, days, weeks, or months. As in Example 1, doses can be administered daily or weekly to treat headache disorders, and doses can be administered daily, weekly, monthly, or semiannually to prevent headache. Treatment can be acute or preventative. Low and very low doses (sometimes referred colloquially as “microdoses”) can also be used, which are generally ⅕ to 1/10 of a standard recreational dose and provides a therapeutic effect without providing a psychedelic effect. Lower doses can be 0.0143 to 0.143 mg/kg (as shown by clinical trial NCT03341689). While oral dosage forms can be preferred, other delivery methods can be used as described below for particular uses, such as intravenous to provide an abortive effect on severe headache attacks. Other psychedelics can be dosed as appropriate such as in micrograms or milligrams.

In treating headache disorders, a single treatment can be administered that provides lasting therapeutic effects. The single treatment can be a single dose or a single pulse regimen as further described below.

For migraines, a single dose can be given, as opposed to current migraine treatments that require daily or other frequent administration. Example 2 below shows that a single low dose of psilocybin had lasting therapeutic effects. Additional follow up doses can also be administered weekly, monthly, or yearly if needed. While psilocybin has been used by individuals for migraines in the past, the dose and regimen have not been accurate or controlled, and the effects found herein have not been reported.

The psychedelic administered can reduce migraine headache burden by reducing the number of migraine days per week (such as by 25%, 50%, or 75% reduction), reducing pain severity, reducing migraine abortive use (i.e. reducing other medications or treatments used for migraines), reducing attack-related functional impairment, and increasing the time between migraine attacks in the individual. Each of these effects was demonstrated in Example 2 below with psilocybin.

For cluster headache, a pulse regimen such as a three dose pulse regimen can be administered to the individual, with a 3 to 7 day separation between doses. A second round of the three dose pulse regimen can be administered at a later time point, such as at least three months or at least six months after the first round. Example 3 below shows that the three dose pulse regimen of low dose psilocybin had lasting therapeutic effects. Other pulse regimens can also be followed having more than three doses. While psilocybin has been used by individuals to manage cluster headache in the past, the dose has not been accurate or controlled, and the effects found herein have not been reported.

Psilocybin can also reduce emotional factors that can aggravate headache burden in headache disorders.

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants and vehicles. The compounds can be administered orally, rectally, sublingually, subcutaneously or parenterally including intravenous, intraarterial, intramuscular, intraperitoneally, intratonsillar, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

The doses can be single doses or multiple doses over a period of several days to weeks. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The present invention provides for a method of treating migraine headache, by administering an effective amount of a psychedelic to an individual and reducing migraine headache burden. The psychedelic can be administered as a migraine preventive (i.e., used in a manner to reduce headache burden over an extended period) or acutely during a migraine attack to treat that single attack (i.e. the administering step is performed during a migraine attack as an acute treatment and further includes reducing pain caused to the individual by the migraine attack). The psychedelic can be dosed in any amount as described above, such as a low dose, and administered in any suitable method.

The present invention provides for a method of treating cluster headache in an individual, by administering an effective amount of a composition of a psychedelic to the individual and reducing cluster headache burden. The psychedelic can be administered as a cluster preventive (i.e., used in a manner to reduce headache burden over an extended period) or acutely during a cluster attack to treat that single attack. The psychedelic can be dosed in any amount as described above, such as a low dose, and administered in any suitable method.

The present invention also provides for a method of treating headache disorders, by administering a single treatment of a psychedelic to an individual and providing a long term effect in preventing headaches. As above, the single treatment can be a single dose or a single pulse regimen. Such an effect is not seen with currently available treatments. The psychedelic can be dosed in any amount as described above and administered in any suitable method.

The invention is further described in detail by reference to the following experimental examples. These examples are provided for the purpose of illustration only and are not intended to be limiting unless otherwise specified. Thus, the invention should in no way be construed as being limited to the following examples, but rather, should be construed to encompass any and all variations which become evident as a result of the teaching provided herein.

### Example 1

A medication use survey was performed, with 41 questions pertaining to demographics, headache characteristics, toxic habits, medication use, and efficacy allowing for multiple choice and free-text answers. There were 651 responders, 558 completed, and 496 with a verified diagnosis.

TABLE 1 shows various demographics and characteristics of the responders.

TABLE 1

| | Percentage |
|---|---|
| Gender | |
| Male | 73.8 |
| Female | 26.2 |
| Race | |
| Caucasian | 93.1 |
| Hispanic | 2.6 |
| Black | 1.2 |
| Asian | <1.0 |
| Other | 1.8 |
| Global Region | |
| United States | 62.5 |
| Europe | 11.6 |
| United Kingdom | 11.6 |
| Canada | 5.0 |
| Africa | <1.0 |
| Asia | <1.0 |
| Other | 8.2 |
| Family History (1^{st} degree) | |
| Yes | 15.1 |
| No | 72.4 |
| Unsure | 12.5 |
| Age at onset of cluster headaches (CH) | |
| <21 | 34.2 |
| 21-30 | 30.8 |
| 31-40 | 18.3 |
| 41-50 | 12.3 |
| 51-60 | 3.6 |
| >60 | 1.0 |
| Time from onset to dx | |
| <6 months | 8.2 |
| <1 year | 13.1 |
| 2 years | 17.3 |
| 3-5 years | 22.2 |
| 6-10 years | 19.6 |
| >10 years | 19.6 |
| Subtype | |
| Episodic | 63.1 |
| Chronic | 15.5 |
| Episodic (formerly chronic) | 4.0 |
| Chronic (formerly episodic) | 15.7 |
| Uncertain | 1.6 |
| Laterality | |
| Current episodics (n = 333) | |
| Right | 44.1 |
| Left | 36.0 |
| Current chronics (n = 154) | |
| Right | 40.3 |
| Left | 30.5 |
| Variation in laterality | |
| Hx episodic CH (n = 354) | |
| Within period | |
| Right (some left) | 3.1 |
| Left (some right) | 4.2 |
| Between periods | |
| Right (switch to left) | 6.2 |
| Left (switch to right) | 3.7 |
| Hx chronic CN (n = 176) | |
| Right (some left) | 15.9 |
| Left (some right) | 9.7 |
| Current Drinker | |
| Yes | 79.0 |
| No | 21.0 |
| Frequency of Drinking | |
| Rare (few × month/year) | 41.8 |
| Once weekly | 21.9 |
| Several times weekly | 23.0 |
| 1-2 times daily | 9.7 |
| >2 times daily | 3.6 |
| Alcohol has triggered an attack | |
| Episodics (n = 300) | 86.7 |
| Chronics (n = 138) | 71.7 |
| Smoker | |
| Never | 21.0 |
| Current | 52.4 |
| Quit | 26.6 |
| Volume smoking prior to CH | |
| <1 PPD | 66.2 |
| 1-2 PPD | 32.7 |
| >2 PPD | 1.2 |
| Effects of quitting on attacks | |
| No effect | 86.7 |
| Fewer/less intense | 9.4 |
| More/more intense | 3.9 |

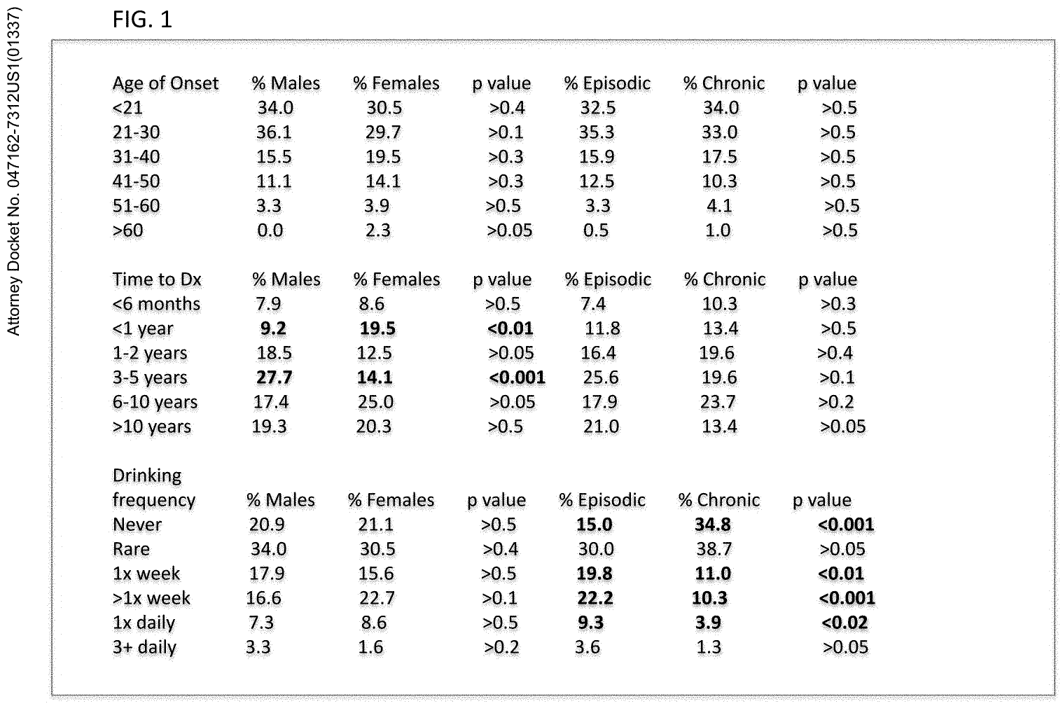

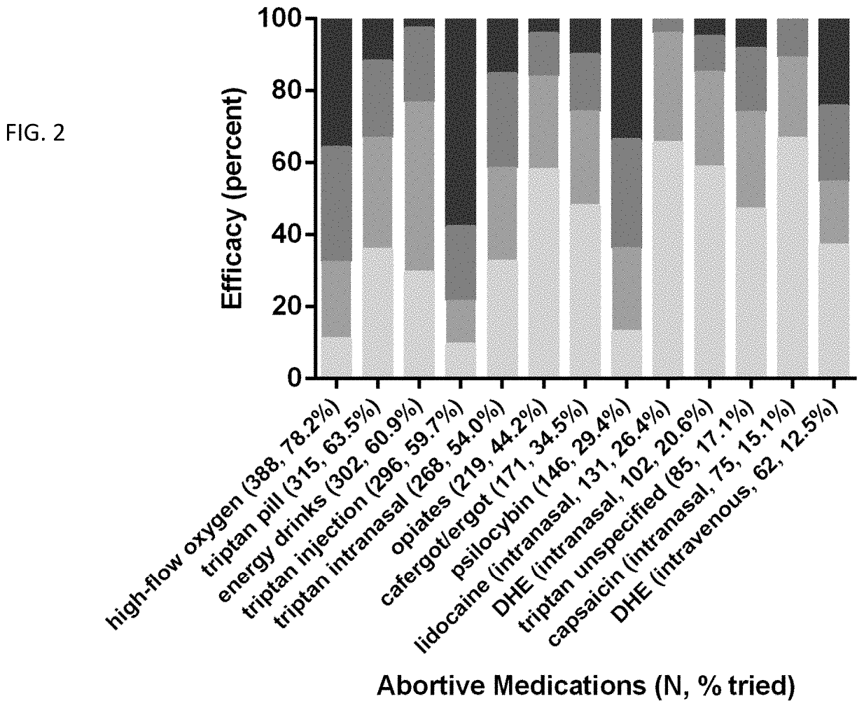

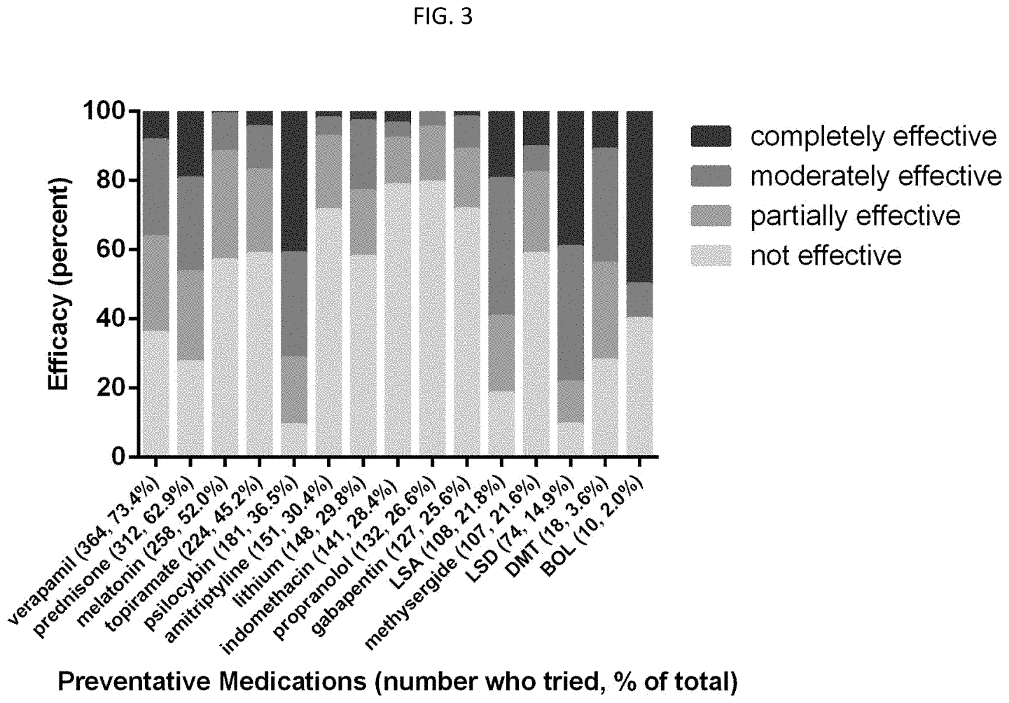

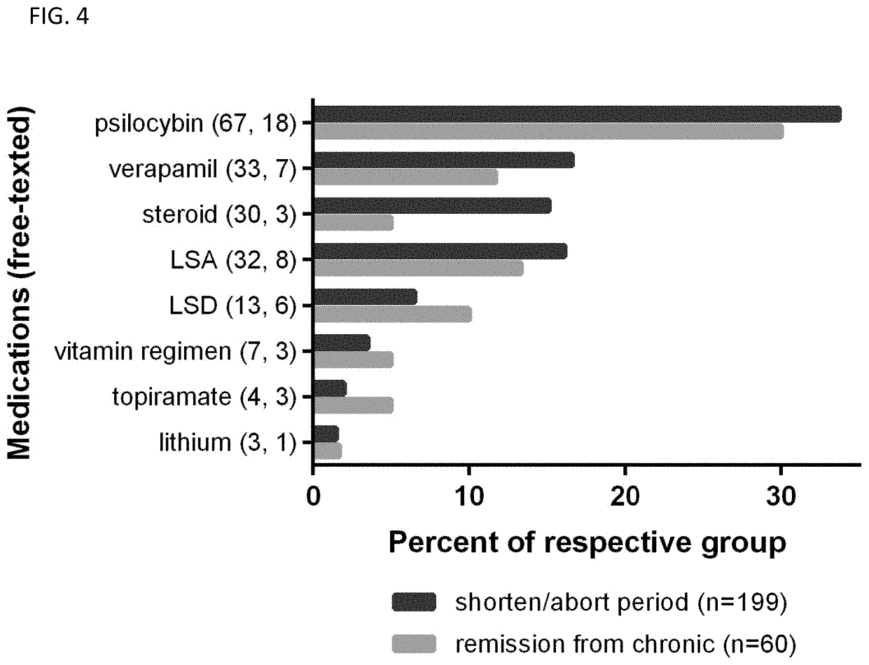

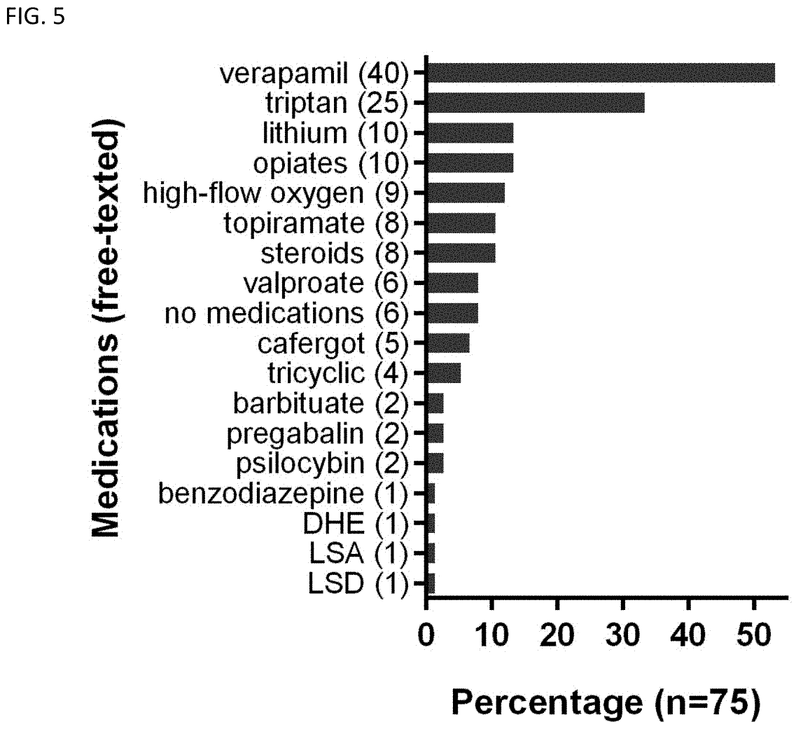

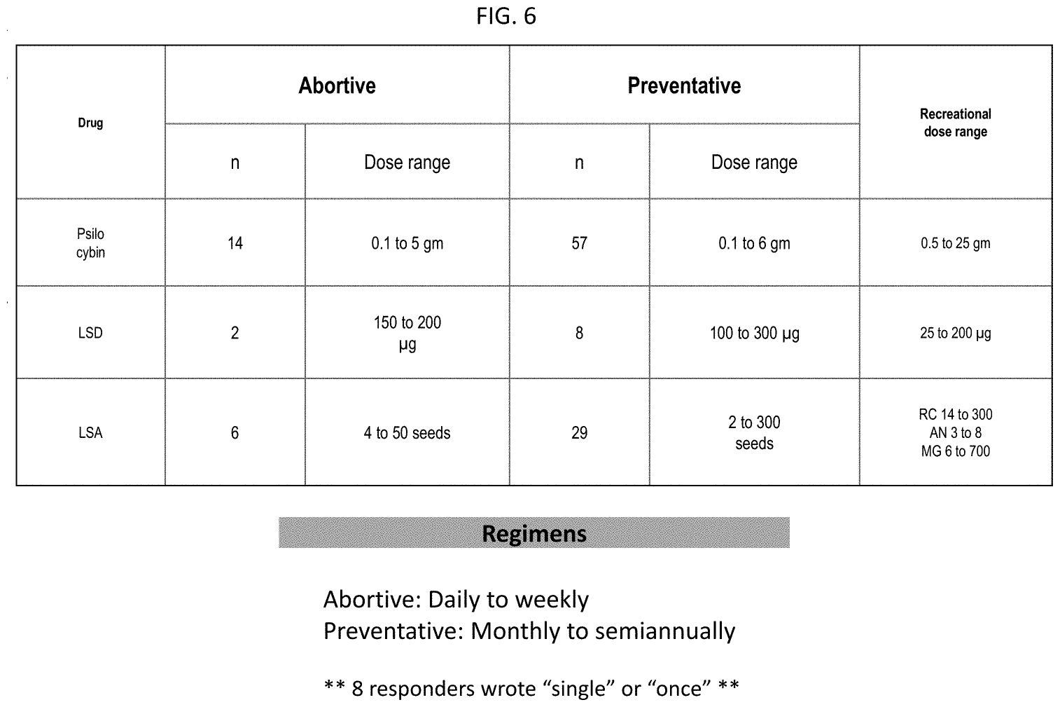

FIG. 1 shows statistics for some of the data gathered from the responders from a survey about cluster headaches. FIG. 2 is a graph of abortive medications with number and percentage of responders who tried versus their reported efficacy. FIG. 3 is a graph of preventative medications with number and percentage of responders who tried versus their reported efficacy. FIG. 4 is a graph comparing medications that effectively affected the cluster period or remission period. FIG. 5 is a graph comparing medications being taken when transforming from episodic to cluster headache. FIG. 6 is a table of medications used and dose ranges. For abortive use, the medications were taken daily to weekly. For preventative use, the medications were taken monthly to semiannually. Eight responders wrote “single” or “once” for their use. The findings from this survey study showed that cluster headache patients reported greater clinical efficacy with psilocybin and other psychedelics as compared to conventional medications.

### Example 2

The purpose of this exploratory study was to investigate the effects of a single low dose of psilocybin in patients with migraine headache in a controlled experiment. Basic information was gathered to assist in the development of future studies considering the effects and mechanism of action of 5-HT2A receptor compounds in migraine and other headache disorders.

Materials and Methods

Regulatory Approvals:

This study was registered on clinicaltrials.gov (NCT03341689) with approvals from the Human Studies Subcommittee of Veterans Affairs Connecticut Healthcare System (VACHS) and the Human Investigations Committee of Yale University School of Medicine. The study was conducted under an approved Investigational New Drug application (#124,874) with the US Food & Drug Administration, under a Schedule 1 license (author DCD). Psilocybin was provided by author NC from the University of Wisconsin. Weight-based capsules of psilocybin 0.143 mg/kg and matching placebo [microcrystalline cellulose; obtained from Fagron (St. Paul, Minn.)] were compounded into identical blue gel capsules by the VACHS Investigational Research Pharmacy for each subject.

Subjects:

Adults (age 21 to 65 years, inclusive) free from serious medical or psychiatric disease with migraine headache as defined by the International Classification of Headache Disorders III-beta and with a frequency of migraine attacks of 2 per week or more were eligible to participate in this study. Among the excluded medical conditions were uncontrolled hypertension, coronary artery disease, cardiac arrhythmia, cerebrovascular disease, and serious central or peripheral nervous system or spinal disease (e.g., multiple sclerosis, amyotrophic lateral sclerosis). Psychotic or manic disorders in the subject or a first degree relative was also excluded, as were substance abuse within the past 3 months and any prior serious adverse event with psilocybin, LSD, or related compounds (i.e., mescaline). Prior exposure to psilocybin or related compounds through recreational or medicinal use or through participation in other research studies was not excluded, although any use in the past 3 months was prohibited. Alcohol use within one week of the first experimental test day was prohibited. Caffeine and nicotine were not restricted. Subjects were required to be free from serotonergic antidepressants (selective serotonin reuptake inhibitors, serotonin and norepinephrine reuptake inhibitors, monoamine oxidase inhibitors, tricyclics) for at least 6 weeks, serotonergic antiemetics (i.e., ondansetron) for at least 2 weeks, and vasoconstrictive medications (i.e., pseudoephedrine) for at least 5 half-lives of said medication. Triptans were permitted, but no more than twice weekly and not within 5 half-lives of said triptan before each test day and not within 5 half-lives of psilocybin (15 hours) after each test day.

Recruitment and Screening:

Subjects were recruited from the local community, headache centers, online headache websites, and word of mouth. Interested candidates were informed of the study and pre-screened over the telephone. If candidates passed the pre-screen based on the study criteria, they were invited for a full evaluation to assess eligibility. This included a medical history, physical examination, laboratory tests (hematology, serum chemistry profile, liver and thyroid studies, urinalysis, urine toxicology, urine pregnancy, electrocardiogram), structured clinical interview for the Diagnostic and Statistical Manual of Mental Disorders, 4th Edition, personality assessment and verbal intelligence quotient test. Subjects' physicians were contacted to verify migraine headache diagnosis and inquire about medical, psychiatric, and substance use history; written consent for this physician contact was required for study participation. Written consent was also obtained in order to speak with a family member or friend in order to exclude any safety concerns for study participation.

Subject Preparation:

During the multi-stage screening process, study procedures and the physiological and psychological effects of psilocybin were repeatedly described. Subjects were also quizzed on study procedures, the expected effects of psilocybin, and emergency contacts.

Assessment of Headache Burden:

Subjects were instructed to maintain a headache diary starting two weeks before the first experimental session until two weeks after the second experimental session. Subjects documented every headache attack, migraine (with associated migrainous symptoms) or otherwise (without associated migrainous symptoms; not counted as a migraine attack). Associated symptoms—photophobia (light sensitivity), phonophobia (sound sensitivity), nausea/vomiting—as well as attack-related functional impairment were documented using a 0-3 visual analog scale (VAS): 0=none, 1=mild, 2-moderate, 3=severe. The abortive medications taken and their effects were also recorded in the diary. Only the 14 days prior to the first experimental session (baseline) and the 14 days after each experimental session (inclusive) were counted in the final analysis.

Experimental Sessions:

Subjects completed two experimental sessions, separated by at least 14 days, which were conducted in the Neurobiological Studies Unit (NSU) at VACHS. Subjects reported to the NSU at 08:00; urine drug, urine pregnancy (when applicable), and alcohol breathalyzer tests were required to be negative in order to proceed. After a standard light breakfast, an intravenous line was placed and baseline measures were collected. Subjects typically received the drug capsule between 08:30 and 09:30. In the first experimental session, all subjects received an oral placebo capsule and in the second experimental session, all subjects received an identically appearing oral psilocybin capsule. In this design, each subject acted as his own control and placebo was given first so that the potential long-term effects of psilocybin did not interfere with placebo treatment. A standard blinding procedure in which drug dose and order of administration were unknown to subjects and research staff was applied. This blinding procedure was approved by both VA and Yale review boards.

Blood pressure, heart rate, and peripheral oxygenation were measured at baseline and throughout experimental sessions. Acute headache pain and associated symptoms (if present) and drug effects (overall, anxiety/fear, sleepiness/sedation, nausea, joy/intense happiness, peace/harmony) were self-reported on a 0-3 scale (0=none, 1=minimal, 2=moderate, 3=definite) at baseline and throughout experimental sessions. Psychedelic effects were self-reported at the end of experimental sessions using the validated 5-Dimensional Altered States of Consciousness (5D-ASC) scale. Subjects were discharged from the NSU at 6 hours after capsule ingestion or once physiological and psychological drug effects had resolved. Only one subject remained in the NSU for an additional 60 minutes until drug effects resolved. Subjects were not allowed to drive themselves after experimental sessions. Emergency contacts, including 24-hour/7-day psychiatry services, were provided to all subjects.

Follow-Up:

Telephone follow-up was performed the day after and weekly for two weeks after each experimental session, and at 2 and 3 months after the second experimental session. After all subjects completed study procedures, subjects were contacted by phone by a study investigator and told what they had received on each experimental test day and the blinding procedure was also revealed.

Statistical Analysis

A total of twelve subjects was sought for this exploratory study. For within-subjects analyses with a two-tailed α=0.05, 12 subjects would provide 80% statistical power to detect large effects (d′=0.9). A total of 69 potential subjects was pre-screened; 14 underwent secondary screening and 12 subjects underwent study procedures. Ten subjects were included in the final analysis. Two subjects were excluded from final analysis; one was unable to participate in the second experimental session for scheduling conflicts and one subject's diary was not usable.

Statistical analyses were performed using SAS, version 9.4 (SAS Institute Inc., Cary, N.C.) and figures were produced with GraphPad Prism (GraphPad Software Inc., La Jolla, Calif.). All statistical tests were two-sided with an overall pre-hypothesis alpha threshold of 0.05. Measures of error are shown as standard error about the mean (SEM). The primary clinical outcome measure was change in weekly migraine days (WMD) from baseline. This and the secondary measures of migraine burden—changes from baseline in weekly migraine attacks (WMA), pain severity, light sensitivity severity, sound sensitivity severity, nausea/vomiting severity, attack-related functional impairment, and weekly migraine abortive use—were calculated and compared via paired t-test. The time to the next two migraine attacks were also compared between placebo and psilocybin via paired t test. Acute effects of drug administration on mean arterial pressure (MAP), heart rate, peripheral oxygenation, and general drug effects were analyzed via 2-way repeated measures ANOVA with post-hoc Fisher least significant difference (LSD) test (when the interaction existed). Psychotropic effects as measured by the 5D-ASC scale were calculated as a percent of the total possible score 21, 22, as well as the percent possible score of each of the scale's five dimensions—oceanic boundlessness (OBN), dread of ego dissolution (DED), visionary restructuralization (VRS), acoustic alterations (AUA), and vigilance reduction (VIR). Percent possible scores were then compared between placebo and psilocybin via paired t-test. In order to test the assumption that psychotropic effects are unrelated to effects on migraine activity, the maximum rating of general drug effects and the percent total possible 5D-ASC scale score were correlated to the percent change in WMD after psilocybin administration. The number of adverse events (AEs) were compared between placebo and psilocybin using inference on proportions.

Results

Demographics, Headache Characteristics, and Substance Use

Seven females and three males were included in the final analysis. The average age was 40.5 (4.4) years with a range from 23 to 63 years. The average age of onset of migraine headache was 18.7 (2.9) years. All subjects endorsed migraine triggers; 100% identified weather changes and 90% identified alcohol as triggers. At the time of enrollment, only one subject indicated that they were satisfied with their current migraine treatment regimen and all subjects indicated that they would at least consider a new migraine treatment if it were available. All subjects had a history of consuming alcohol, but only 70% were current drinkers. Two subjects had previously tried psilocybin. One subject had a history of substance abuse (over 10 years prior), involving alcohol and cocaine (in remission at the time of study participation).

Migraine Headache Burden after Drug Administration

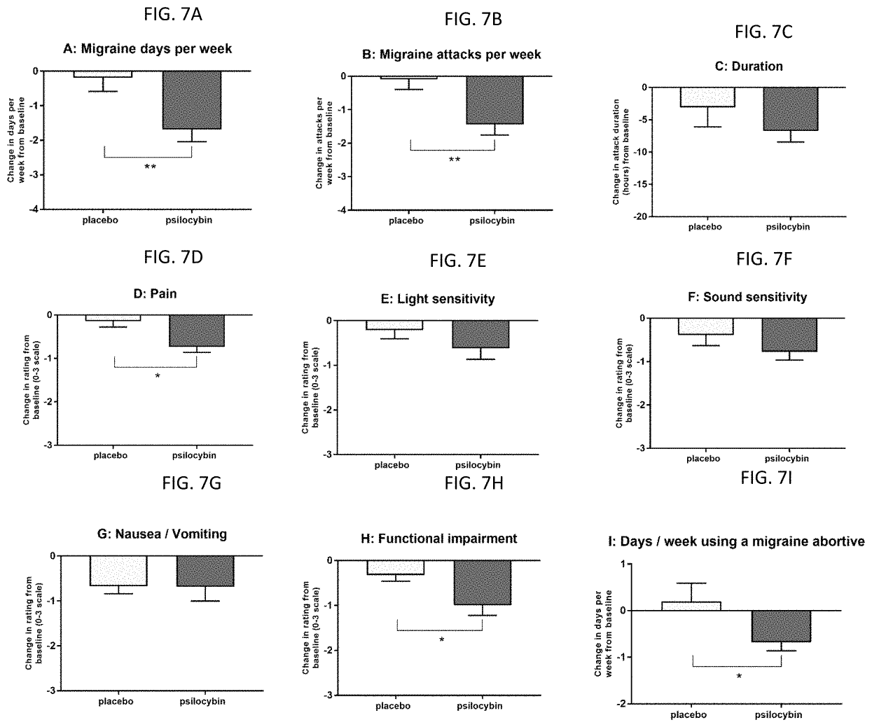

Weekly migraine days (WMD): Psilocybin significantly reduced WMD from baseline as compared to placebo. The change in WMD from baseline was −0.15 (0.43) days/week after placebo and −1.65 (0.39) days/week after psilocybin (P=0.0027, df=9; paired t-test; FIG. 7A). The percentage of subjects who had 25%, 50%, and 75% reductions in WMD was 20%, 20%, and 0% after placebo, respectively, and 80%, 50%, and 30% after psilocybin, respectively.

Additional measures of migraine burden: Psilocybin significantly reduced WMA, pain severity, and weekly migraine abortive use from baseline as compared to placebo. Changes from baseline were significantly different between drug in WMA [placebo −0.05 (0.35) attacks/week, psilocybin −1.40 (0.36) attacks/week; P=0.0039, df=9; FIG. 7B], pain severity [placebo −0.11 (0.16), psilocybin −0.70 (0.16); P=0.0109, df=7; FIG. 7D], attack-related functional impairment [placebo −0.29 (0.17), psilocybin −0.96 (0.26); P=0.0235, df=9; FIG. 7H], and weekly migraine abortive use [placebo +0.20 (0.39) abortives/week, psilocybin −0.65 (0.21) abortives/week; P=0.0140, df=9; FIG. 7I] (all paired t-tests). There were no significant differences in the effects of placebo and psilocybin in migraine attack duration or associated symptom (light sensitivity, sound sensitivity nausea/vomiting) ratings (FIGS. 7E, 7F, 7G).

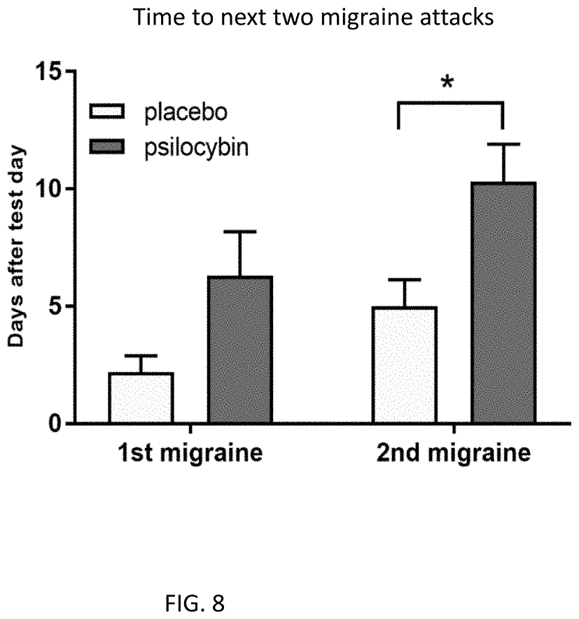

Time to first and second migraine attacks: The difference in the time to the first migraine attack after drug administration was not significant between placebo [2.20 (0.70) days] and psilocybin [6.30 (1.88) days; P=0.0554; paired t-test, df=9; paired t-test; FIG. 8]. Given that psilocybin is known to induce headaches in the short-term (see adverse events), the time to the second migraine attack was also measured. The difference in the time to the second migraine attack between placebo [5.00 (1.13) days] and psilocybin [10.30 (1.61) days] was significant (P=0.0119, df=9; paired t-test; FIG. 8). Of note, there were ceiling effects involved in these measures, as 2 subjects had only one migraine attack and 2 had no migraine attacks after psilocybin administration. When no migraine attacks occurred for the outcome measured, “15 days” was used as the time to that attack.

Acute Effects of Drug Administration

Physiologic effects: There was a significant drug x time interaction for MAP over the experimental test day (P<0.0073, F=2.52, df=10; repeated measures 2-way ANOVA). Post-hoc analysis (Fisher LSD) revealed a significant increase in MAP with psilocybin administration starting at 45 minutes until 4 hours after capsule ingestion. The maximum increase in MAP over placebo was 12.2 (4.7) mmHg at 1.5 hours after capsule ingestion. There were no significant interactions on heart rate or peripheral oxygenation during experimental sessions.

Psychotropic effects: During experimental test sessions, both placebo and psilocybin elicited general drug effects, with significant drug x time interactions for overall drug effects (P=0.0005, F=3.74, df=10) and peace/harmony (P=0.0352, F=2.15, df=10; repeated measures 2-way ANOVA). Post-hoc analysis revealed a significant change in overall drug effects between 1 and 4 hours after capsule ingestion. The feeling of peace/harmony was only significantly elevated over placebo at 6 hours after capsule ingestion. Significant interactions were lacking for sleepiness/sedation, anxiety/fear, nausea, and joy/intense happiness.

Both placebo and psilocybin elicited classic psychedelic effects as measured by the 5D-ASC scale, which was completed by subjects at the end of each experimental test day. The percent possible score for the total scale score was significantly higher after psilocybin [19.35% (7.55)] as compared to placebo [3.08% (1.80); P=0.0263, df=9; paired t-test]. The individual dimensions of the 5D-ASC scale did not survive multiple comparisons to reveal significant differences between placebo and psilocybin (interaction P=0.0741; repeated measures 2-way ANOVA).

Relationship Between Acute Drug Effects and Migraine Headache Burden

The maximum overall drug effect rating during psilocybin exposure did not correlate with the percent change from baseline in WMD (R=0.405). In fact, the three subjects with the highest overall drug effect score had the least reduction in WMD. The percent total 5D-ASC scale score during psilocybin exposure also did not correlate with the percent change from baseline in WMD (R=0.412). Again, the highest percent total 5D-ASC scale score was actually seen in a subject with minimal reduction in WMD.

Adverse Events

There were no serious or unexpected AEs in this study. During experimental sessions, lightheadedness and generalized tension/sore muscles were reported by some subjects after either placebo or psilocybin, but the numbers between drugs were not significant. Confusion was reported by one subject after placebo. Nausea (n=4) and anxiety (n=3) were only reported after psilocybin (zero for placebo; nausea P<0.01; anxiety P<0.04; inference on proportions). Tingling/paresthesia, cold/shivering, dry mouth, and acute migraine were reported by one subject each after psilocybin. In the 24 hours following the start of experimental sessions, both placebo and psilocybin administration were followed by tension/sore muscles, general headache attack, and migraine attack in some subjects, but there were no significant differences between placebo and psilocybin. All adverse events were transient and self-limiting. No subjects withdrew from the study due to an AE.

Discussion

This exploratory double-blind, placebo-controlled, cross-over investigation showed significant reductions in migraine headache burden in the two weeks after administration of a single low oral dose of psilocybin. This is the first controlled study reporting therapeutic effects of psilocybin in migraine headache. In addition, the administration of a single low oral dose of psilocybin in patients with migraine headache in the experimental setting was well-tolerated and lacked any serious or unexpected AEs.

The findings from this study complement research in past decades with psilocybin, LSD, and related drugs that have demonstrated lasting beneficial effects in depression, anxiety, and substance abuse after limited exposure to the drug. The lasting therapeutic effect after the single administration of an oral agent reported in this study is a novel finding for migraine headache. This contrasts with existing preventive migraine therapies which necessitate repeated, daily drug administration (e.g., topiramate) or treatments which remain in the body long after administration (e.g., monoclonal antibodies). The long-lasting effects of psilocybin in the current study are not dissimilar to transitional migraine treatments, such as corticosteroids, which are administered in oral pulses of various duration or dihydroergotamine (DHE), which is administered as a thrice daily five-day intravenous or subcutaneous injection regimen. It is notable that DHE is also an indoleamine agent with activity at the 5-HT2A receptor, in additional to several other receptors. Whether a shared mechanism of action for migraine headache burden reduction exists between psilocybin and DHE or psilocybin and corticosteroids will require further study.

The current study did not find that psychotropic effects correlated with the change in migraine headache burden, suggesting that the sustained effects of psilocybin in migraine headache are independent from acute changes in sensation and perception. Indeed, some subjects with large decreases in WMD had very low 5D-ASC scale scores, indicating minimal sensory and perceptual alterations during experimental sessions. These observations are consistent with survey studies reporting that sub-hallucinogenic doses of psilocybin and LSD provide relief in headache disorders. Furthermore, a congener of LSD, 2-bromo-lysergic acid diethylamide (BOL-148 or BOL), which has greatly reduced psychotropic effects, is also reported to have medicinal effects in cluster and other headache disorders. Collectively, these findings suggest dissociation between the acute psychotropic effects and the sustained therapeutic action of psilocybin and other 5-HT2A receptor agonist compounds in headache disorders.

Limitations

This study has several limitations. The sample size is small, though appropriate for an exploratory investigation. Strong statistical significance and large effect sizes validate the findings in this small sample. All subjects were Caucasian and had relatively high starting headache burden and thus were not representative of the general migraine population. Subjects were included meeting criteria for either episodic or chronic migraine headache in the present study. In roughly identifying subjects with headache attacks per week as having chronic migraine headache, subgroup analysis did not reveal significant differences between chronics (n=5) and episodics (n=5; data not shown). There were also no obvious age or sex differences (data not shown), though future, adequately powered studies will be necessary to conclusively determine if any such differences exist. Lastly, psilocybin induced general, physiological and psychotropic effects, which might suggest to subjects what treatment they received. Blinding of agents such as psilocybin is challenging, however these effects were also reported after placebo administration, indicating an effective blinding procedure.

### Conclusion

In the first controlled investigation of psilocybin in a neurological condition, sustained reductions were demonstrated in migraine headache burden after the administration of a single low oral dose. This preliminary study supports the viability of this drug as an experimental agent in migraine headache and showed that with careful recruitment, screening, preparatory, monitoring, and follow-up procedures, low dose psilocybin can safely be administered to patients with migraine headache in the research setting. This study also represents a new arm in the field of select 5-HT2A receptor compounds, offering a new perspective on the unique abilities of this drug class.

### Example 3

In this exploratory study, a patient-informed regimen (a low dose of psilocybin (between 1-2 grams dried P. cubensis taken three times, approximately 5 days apart each)) was studied in patients with cluster headache in a controlled, laboratory setting. The information gained from this study serves to (1) verify longstanding anecdotal reports and (2) design larger, more definitive studies examining the safety and efficacy of psilocybin in cluster headache.

Materials and Methods

Regulatory Approvals:

This study was registered on clinicaltrials.gov (NCT02981173) with approvals from the Human Studies Subcommittee of Veterans Affairs Connecticut Healthcare System (VACHS) and the Human Investigations Committee of Yale University School of Medicine. The study was conducted under an approved Investigational New Drug application (#124,874) with the US Food & Drug Administration, under a Schedule 1 license (author DCD). Psilocybin was provided by author NC from the University of Wisconsin. Weight-based capsules of psilocybin 0.143 mg/kg and matching placebo (microcrystalline cellulose; obtained from Fagron (St. Paul, Minn.)) were compounded into identical blue gel capsules by the VACHS Investigational Research Pharmacy for each subject.

Subjects:

Adults (age 21 to 65 years, inclusive) with cluster headache as defined by the International Classification of Headache Disorders III-beta 8 were eligible to participate in this study. Both chronic and episodic cluster headache subtypes were eligible, however certain conditions were required to ensure proper interpretation of the study intervention. For both chronic and episodic subjects, an attack frequency of approximately 1 attack per day or more was required. For episodic subjects, the typical cluster period was required to last approximately 2 months or more and study participation could only take place when at least 6 weeks of the period was anticipated to remain. Excluded medical conditions included, but were not limited to, uncontrolled hypertension, coronary artery disease, cardiac arrhythmia, cerebrovascular disease, and serious central or peripheral nervous system or spinal disease. Excluded psychiatric conditions included, but were not limited to, psychotic or manic disorders in the subject or a first degree relative and substance abuse within the past 3 months. Any prior serious adverse event with psilocybin, LSD, or related compounds (i.e., mescaline) was also exclusionary. Prior exposure to psilocybin or related compounds through recreational or medicinal use or through participation in other research studies was not excluded, although any use in the past 3 months was prohibited. Alcohol use within one week of the first experimental test day was prohibited. Caffeine and nicotine were not restricted. Subjects were required to be off serotonergic antidepressants (selective serotonin reuptake inhibitors, serotonin and norepinephrine reuptake inhibitors, monoamine oxidase inhibitors, tricyclics) for at least 6 weeks and serotonergic antiemetics (i.e., ondansetron) for at least 2 weeks from the first test day. Vasoconstrictive medications (i.e., sumatriptan, pseudoephedrine) could be used during the study period, but not within 5 half-lives of said medication and not within 5 half-lives of psilocybin (15 hours) after each test day. Triptans were also not allowed more than twice weekly.

Recruitment and Screening:

Subjects were recruited from the local community, headache centers, online headache websites, and word of mouth. Interested candidates were informed of the study and pre-screened over the telephone. If candidates passed the pre-screen based on the study criteria, they were invited for a full evaluation to assess eligibility. This included a medical history, physical examination, laboratory tests (hematology, serum chemistry profile, liver and thyroid studies, urinalysis, urine toxicology, urine pregnancy, electrocardiogram), structured clinical interview for the Diagnostic and Statistical Manual of Mental Disorders, 4th Edition, personality assessment and verbal intelligence quotient test. Subjects' physicians were contacted to verify cluster headache diagnosis and inquire about medical, psychiatric, and substance use history; written consent for this physician contact was required for study participation. Written consent was also obtained in order to speak with a family member or friend in order to exclude any safety concerns for study participation.

Subject Preparation:

During the multi-stage screening process, study procedures and the physiological and psychological effects of psilocybin were repeatedly described. Subjects were also quizzed on study procedures, the expected effects of psilocybin, and emergency contacts.

Assessment of Headache Burden:

Subjects were instructed to maintain a headache diary starting 2 weeks before until 8 weeks after the first experimental session. Subjects documented every cluster attack, including date, onset, offset, pain intensity (0-10 visual analog scale (VAS): 0=none, 1=minimal, 5=moderate, 9=severe, 10=worst imaginable). The abortive treatments taken and their effects were also recorded in the diary. Only the 14 days prior to the first experimental session (baseline) and the 56 days after the first experimental session (inclusive) were counted in the final analysis.

Experimental Design:

Subjects were randomized to receive a 3-dose pulse regimen of either psilocybin (0.143 mg/kg) or placebo (microcrystalline cellulose) and received the same drug on each of 3 test days. Each test day was separated by 5±2 days. No less than 6 months after the start of their first pulse regimen, subjects who still qualified for study participation (i.e., still having attacks, entering a new cluster period) were invited back for a second round of experimental treatment, this time without the possibility of receiving placebo. A standard blinding procedure in which multiple possible drug doses were included in the randomization was approved by both VA and Yale review boards and applied to both the first and second rounds of experimental treatment so that subjects would not know their group assignment.

Experimental Sessions:

Sessions were conducted in the Neurobiological Studies Unit (NSU) at VACHS. Subjects reported to the NSU at 08:00; urine drug, urine pregnancy (when applicable), and alcohol breathalyzer tests were required to be negative in order to proceed. After a standard light breakfast, an intravenous line was placed and baseline measures were collected. Subjects typically received the drug capsule between 08:30 and 09:30. Blood pressure, heart rate, and peripheral oxygenation were measured at baseline and throughout experimental sessions. Drug effects (overall, anxiety/fear, sleepiness/sedation, nausea, joy/intense happiness, peace/harmony) were self-reported on a 0-3 scale (0=none, 1=minimal, 2=moderate, 3=definite) at baseline and throughout experimental sessions. Psychedelic effects were self-reported at the end of experimental sessions using the validated 5-Dimensional Altered States of Consciousness (5D-ASC) scale. Subjects were discharged from the NSU at 6 hours after capsule ingestion or once physiological and psychological drug effects had resolved. Subjects were not allowed to drive themselves after experimental sessions. Emergency contacts, including 24-hour/7-day psychiatry services, were provided to all subjects.

Follow-Up:

Telephone follow-up was performed the day after each experimental session and weekly for two weeks and at 2, 3, and 6 months after the last experimental session. After all subjects completed study procedures for the first round of experimental treatment, subjects who declined or did not qualify for a second round were contacted by phone by a study investigator and told what they had received on the test days and the blinding procedure was also revealed. For those subjects who partook in a second round of experimental round of treatment, their drug assignments from both the first and second rounds were not revealed until after all participating subjects completed their second round.

Results

Demographics, Headache Characteristics, and Substance Use

The Psilocybin Pulse Regimen (Administered Once) Reduced Cluster Headache Burden as Compared to Placebo.

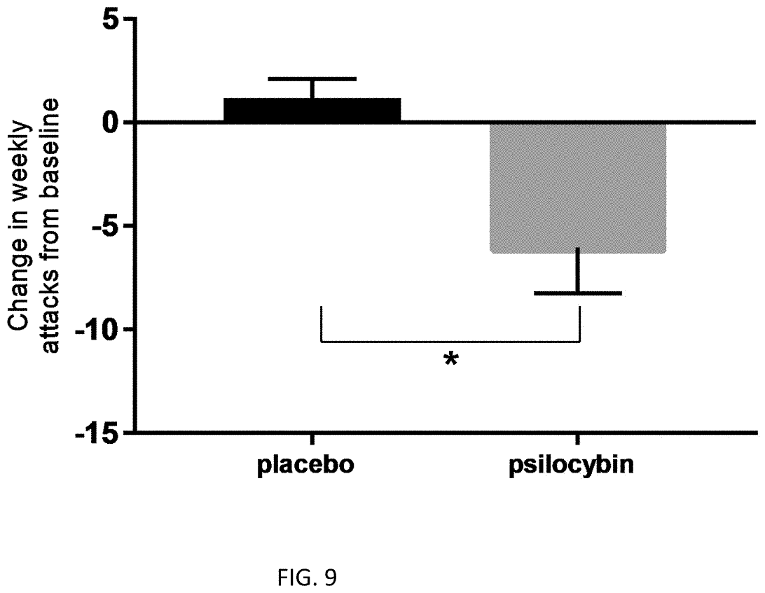

The number of weekly cluster attacks in the placebo group (9.10 (2.41)) rose over the first 3 weeks (10.27 (2.68)), whereas the number of weekly cluster attacks in the psilocybin group (9.30 (1.49)) fell by more than half (3.50 (1.12)). The change in the number of weekly cluster attacks was significantly different between placebo (+1.17 (0.92)) and psilocybin (−6.03 (2.23); P=0.017, paired t-test). The effect size for this change is extremely large at −1.89. This measure combined episodic and chronic patients and was measured at three weeks so as to avoid the potential natural termination of episodic cluster periods in the treatment effects. These results are shown in FIG. 9.

The effect of the psilocybin regimen (administered once) can be seen out to 8 weeks after drug administration.

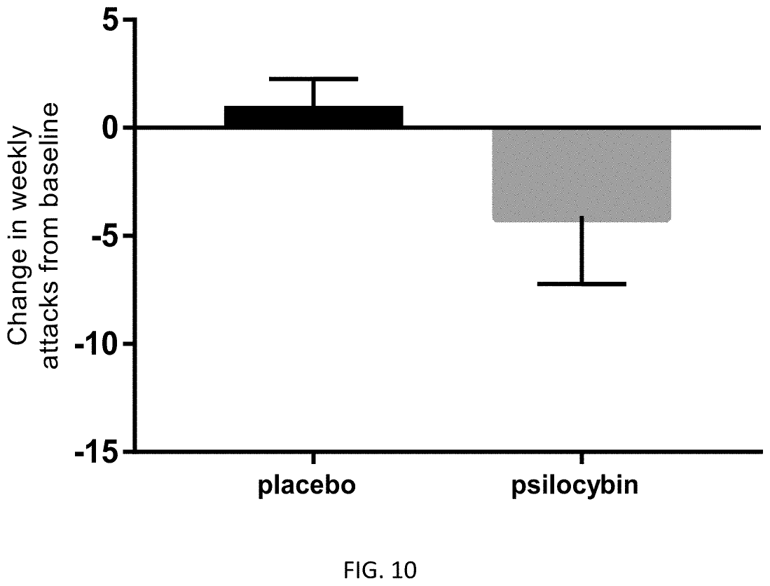

In chronic cluster headache subjects, the effect of treatment can be looked at over a longer term because the confound of natural cycle termination is lacking. The reduction in weekly cluster attacks after psilocybin is seen across the 8-week period of the diary. The number of weekly cluster attacks in the placebo group (8.50 (3.01)) rose across 8 weeks (9.50 (3.45)), whereas the number of weekly cluster attacks in the psilocybin group (8.67 (2.42)) fell by almost half (4.58 (0.77)). The change in the number of weekly cluster attacks approached significance between placebo (−0.37 (1.68)) and psilocybin (−6.08 (2.29); P=0.080, paired t-test). The effect size for this change remains very large at −1.20. These results are shown in FIG. 10.

The Psilocybin Regimen (Administered Once) was Well-Tolerated and Safe.

There were no serious or unexpected adverse events from study participation. The most commonly reported adverse events during acute psilocybin exposure included nausea and anxiety, both of which were self-limiting. In following up with subjects in the 6 months after study participation, no lasting physical or psychological changes were reported.

Discussion

These preliminary findings strongly support a therapeutic effect of the psilocybin pulse regimen on cluster headache burden. Continued study of psilocybin in cluster headache is warranted, particularly as several questions about how this drug might be applied in practice remain.

### CONCLUSION

In the first controlled investigation of psilocybin in cluster headache, reduction in attack frequency with the administration of a 3-dose pulse regimen has been demonstrated.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Many modifications and variations of the present invention are possible in light of the above teachings. It is, therefore, to be understood that within the scope of the appended claims, the invention can be practiced otherwise than as specifically described.

### Enumerated Embodiments

The following enumerated embodiments are provided, the numbering of which is not to be construed as designating levels of importance:

Embodiment 1 provides a method of treating headache disorders, the method comprising: administering an effective amount of a composition comprising a psychedelic to an individual in need thereof; and treating the headache disorder.

Embodiment 2 provides them method of embodiment 1, wherein the psychedelic is selected from the group consisting of psilocybin, lysergic acid diethylamide (LSD), mescaline, dimethyltryptamine (DMT), 2,5-dimethoxy-4-iodoamphetamine (DOI), 2,5-dimethoxy-4-bromoamphetamie (DOB), salts thereof, tartrates thereof, analogs thereof, or homologues thereof.

Embodiment 3 provides the method of any one of embodiments 1-2, wherein the headache disorder is selected from the group consisting of migraine, tension-type headache, cluster headache, and secondary headache disorders.

Embodiment 4 provides the method of any one of embodiments 1-3, wherein the method further comprises a step of reducing headache burden by acute treatment of the headache disorder or prevention of the headache disorder.

Embodiment 5 provides the method of any one of embodiments 1-4, wherein said administering step comprises administering 1-50 mg of psilocybin orally.

Embodiment 6 provides the method of any one of embodiments 1-5, wherein said administering step further comprises administering the composition daily, weekly, monthly, or semiannually.

Embodiment 7 provides a method of treating migraine headache, the method comprising: administering an effective amount of a psychedelic to an individual in need thereof; and reducing migraine headache burden.

Embodiment 8 provides the method of embodiment 7, wherein the psychedelic is selected from the group consisting of psilocybin, lysergic acid diethylamide (LSD), mescaline, dimethyltryptamine (DMT), 2,5-dimethoxy-4-iodoamphetamine (DOI), 2,5-dimethoxy-4-bromoamphetamie (DOB), salts thereof, analogs thereof, or homologues thereof.

Embodiment 9 provides the method of any one of embodiments 7-8, wherein said administering step further comprises administering a single dose or single pulse treatment of psychedelic to the individual, and further comprises the step of providing a lasting therapeutic effect.

Embodiment 10 provides the method of any one of embodiments 7-9, wherein said administering step further comprises administering 1-50 mg of psilocybin orally.

Embodiment 11 provides the method of any one of embodiments 7-10, wherein said administering step is further comprises administering 0.143 mg/kg of psilocybin orally.

Embodiment 12 provides the method of any one of embodiments 7-11, further comprising the step of administering a follow up dose of the psychedelic at a time selected from the group consisting of weekly, monthly, and yearly.

Embodiment 13 provides the method of any one of embodiments 7-12, wherein said reducing step further comprises a step selected from the group consisting of reducing the number of migraine days per week, reducing pain severity, reducing migraine abortive use, reducing attack-related functional impairment, and increasing the time between migraine attacks in the individual.

Embodiment 14 provides the method of any one of embodiments 7-13, wherein said reducing step further comprises a step selected from the group consisting of acutely treating migraine headache and preventing migraine headache.

Embodiment 15 provides a method of treating cluster headache, the method comprising: administering an effective amount of a psychedelic to an individual in need thereof; and reducing cluster headache burden.

Embodiment 16 provides the method of embodiment 15, wherein the psychedelic is selected from the group consisting of psilocybin, lysergic acid diethylamide (LSD), mescaline, dimethyltryptamine (DMT), 2,5-dimethoxy-4-iodoamphetamine (DOI), 2,5-dimethoxy-4-bromoamphetamie (DOB), salts thereof, analogs thereof, or homologues thereof.

Embodiment 17 provides the method of any one of embodiments 15-16, wherein said administering step further comprises administering the psychedelic in a three dose pulse regimen with a 3-7 day separation between doses.

Embodiment 18 provides the method of embodiment 17, further comprising the step of administering a second round of the three dose pulse regimen at a later time.

Embodiment 19 provides the method of any one of embodiments 15-18, wherein said administering step further comprises administering 1-50 mg of psilocybin orally.

Embodiment 20 provides the method of any one of embodiments 15-19, wherein said administering step further comprises administering 0.143 mg/kg of the psilocybin orally.

Embodiment 21 provides the method of any one of embodiments 15-20, wherein said administering step further comprises administering the composition daily, weekly, monthly, or semiannually.

Embodiment 22 provides the method of any one of embodiments 15-21, wherein said reducing step further comprises reducing the number of weekly cluster attacks in the individual.

Embodiment 23 provides the method of any one of embodiments 15-22, wherein said reducing step further comprises a step selected from the group consisting of acutely treating cluster headache and preventing cluster headaches.

Embodiment 24 provides a method of treating headache disorders, the method comprising: administering a single treatment of a psychedelic to an individual in need thereof; and providing a long term effect in preventing headaches.

Embodiment 25 provides the method of embodiment 24, wherein the treatment is selected from the group consisting of a single dose and a single pulse regimen.

Embodiment 26 provides the method of any one of embodiments 24-25, wherein the headache disorder is selected from the group consisting of migraine and cluster headache.

Embodiment 27 provides the method of any one of embodiments 24-26, wherein the psychedelic is selected from the group consisting of psilocybin, lysergic acid diethylamide (LSD), mescaline, dimethyltryptamine (DMT), 2,5-dimethoxy-4-iodoamphetamine (DOI), 2,5-dimethoxy-4-bromoamphetamie (DOB), salts thereof, analogs thereof, or homologues thereof.

Embodiment 28 provides the method of any one of embodiments 24-27, wherein said administering step further comprises administering 1-50 mg of psilocybin orally.

Embodiment 29 provides the method of any one of embodiments 24-28, wherein said administering step further comprises administering 0.143 mg/kg of the psilocybin orally.

What is claimed is:

1. A method of treating headache disorders, the method comprising:
administering an effective amount of a composition comprising a psychedelic to an individual in need thereof; and
treating the headache disorder.

2. The method of claim 1, wherein the psychedelic is selected from the group consisting of psilocybin, lysergic acid diethylamide (LSD), mescaline, dimethyltryptamine (DMT), 2,5-dimethoxy-4-iodoamphetamine (DOI), 2,5-dimethoxy-4-bromoamphetamie (DOB), salts thereof, analogs thereof, or homologues thereof.

3. The method of claim 1, wherein the headache disorder is selected from the group consisting of migraine, tension-type headache, cluster headache, and secondary headache disorders.

4. The method of claim 1, wherein the method further comprises a step of reducing headache burden by acute treatment of the headache disorder or prevention of the headache disorder.

5. The method of claim 1, wherein said administering step comprises administering 1-50 mg of psilocybin orally.

6. The method of claim 1, wherein said administering step further comprises administering the composition daily, weekly, monthly, or semiannually.

7. A method of treating migraine headache, the method comprising:
administering an effective amount of a psychedelic to an individual in need thereof; and
reducing migraine headache burden.

8. The method of claim 7, wherein the psychedelic is selected from the group consisting of psilocybin, lysergic acid diethylamide (LSD), mescaline, dimethyltryptamine (DMT), 2,5-dimethoxy-4-iodoamphetamine (DOI), 2,5-dimethoxy-4-bromoamphetamie (DOB), salts thereof, analogs thereof, or homologues thereof.

9. The method of claim 7, wherein said administering step further comprises administering a single dose or single pulse treatment of psychedelic to the individual, and further comprises the step of providing a lasting therapeutic effect.

10. The method of claim 7, wherein said administering step further comprises administering 1-50 mg of psilocybin orally.

11. The method of claim 10, wherein said administering step further comprises administering 0.143 mg/kg of psilocybin orally.

12. The method of claim 7, further comprising the step of administering a follow up dose of the psychedelic at a time selected from the group consisting of weekly, monthly, and yearly.

13. The method of claim 7, wherein said reducing step further comprises a step selected from the group consisting of reducing the number of migraine days per week, reducing pain severity, reducing migraine abortive use, reducing attack-related functional impairment, and increasing the time between migraine attacks in the individual.

14. The method of claim 7, wherein said reducing step further comprises a step selected from the group consisting of acutely treating migraine headache and preventing migraine headache.

15. A method of treating cluster headache, the method comprising:
administering an effective amount of a psychedelic to an individual in need thereof; and
reducing cluster headache burden.

16. The method of claim 15, wherein the psychedelic is selected from the group consisting of psilocybin, lysergic acid diethylamide (LSD), mescaline, dimethyltryptamine (DMT), 2,5-dimethoxy-4-iodoamphetamine (DOI), 2,5-dimethoxy-4-bromoamphetamie (DOB), salts thereof, analogs thereof, or homologues thereof.

17. The method of claim 15, wherein said administering step further comprises administering the psychedelic in a three dose pulse regimen with a 3-7 day separation between doses.

18. The method of claim 17, further comprising the step of administering a second round of the three dose pulse regimen at a later time.

19. The method of claim 15, wherein said administering step further comprises administering 1-50 mg of psilocybin orally.

20. The method of claim 19, wherein said administering step further comprises administering 0.143 mg/kg of the psilocybin orally.

21. The method of claim 15, wherein said administering step further comprises administering the composition daily, weekly, monthly, or semiannually.

22. The method of claim 15, wherein said reducing step further comprises reducing the number of weekly cluster attacks in the individual.

23. The method of claim 15, wherein said reducing step further comprises a step selected from the group consisting of acutely treating cluster headache and preventing cluster headaches.

24. A method of treating headache disorders, the method comprising:
administering a single treatment of a psychedelic to an individual in need thereof; and
providing a long term effect in preventing headaches.

25. The method of claim 24, wherein the treatment is selected from the group consisting of a single dose and a single pulse regimen.

26. The method of claim 24, wherein the headache disorder is selected from the group consisting of migraine and cluster headache.

27. The method of claim 24, wherein the psychedelic is selected from the group consisting of psilocybin, lysergic acid diethylamide (LSD), mescaline, dimethyltryptamine (DMT), 2,5-dimethoxy-4-iodoamphetamine (DOI), 2,5-dimethoxy-4-bromoamphetamie (DOB), salts thereof, analogs thereof, or homologues thereof.

28. The method of claim 24, wherein said administering step further comprises administering 1-50 mg of psilocybin orally.

29. The method of claim 28, wherein said administering step further comprises administering 0.143 mg/kg of the psilocybin orally.
